(19) United States
(12) Patent Application Publication
(10) Pub. No.: US 2016/0213669 A1
(43) Pub. Date: Jul. 28, 2016

(54) USE OF N-(4-((3-(2-AMINO-4-PYRIMIDINYL)-2-PYRIDINYL)OXY)PHENYL)-4-(4-METHYL-2-THIENYL)-1-PHTHALAZINAMINE IN COMBINATION WITH HISTONE DEACETYLASE INHIBITORS FOR TREATMENT OF CANCER

(71) Applicant: THE JOHNS HOPKINS UNIVERSITY, Baltimore, MD (US)

(72) Inventors: Michael Carducci, Baltimore, MD (US); Sushant Kachhap, Baltimore, MD (US); Channing Paller, Bethesda, MD (US)

(21) Appl. No.: 15/088,799

(22) Filed: Apr. 1, 2016

Related U.S. Application Data
(63) Continuation of application No. 14/388,949, filed on Sep. 29, 2014, now abandoned.

(51) Int. Cl. A61K 31/506; A61K 31/167; A61K 45/06

(52) U.S. Cl. CPC A61K 31/506; A61K 45/06; A61K 31/167

(57) ABSTRACT

The present invention relates to methods of using AMG 900, a small molecule pan aurora kinase inhibitor, in combination with histone deacetylase (HDAC) inhibitor for the treatment of cancer, including solid tumors, hematologically derived tumors and the like. The invention further provides pharmaceutical compositions for administering the cancer therapeutic agents in combination.

### RELATED APPLICATIONS

This application claims the benefit of U.S. Provisional Patent Application No. 61/618,090 filed on Mar. 30, 2012, which w incorporated herein by reference in its entirety.

### STATEMENT OF GOVERNMENTAL INTEREST

This invention was made with U.S. government support under grant nos. P30-C006973-41S2 and P50CA05836. The U.S. government has certain rights in the invention.

### FIELD OF THE INVENTION

The present invention relates to the use of N-(4-((3-(2-amine-4-pyrimidinyl)-2-pyridinyl)oxy)phenyl)-4-(4-methyl-2-thienyl)-1-phthalazinamine in combination with histone deacetylase inhibitors (HDAC inhibitors) for the treatment of cancer.

### BACKGROUND OF THE INVENTION

Cancer is one of the most widespread diseases affecting Mankind, and a leading cause of death worldwide. In the United States alone, cancer is the second leading cause of death, surpassed only by heart disease. Cancer is often characterized by deregulation of normal cellular process or unregulated cell proliferation. Cells that have been transformed to cancerous cells tend to proliferate in an uncontrolled and unregulated manner leading to, in some cases, metastasis or the spread of the cancer. Deregulation of the cell proliferation could result from the modification to one or more genes, responsible for the cellular pathways that control cell-cycle progression. Or it could result from DNA modifications (including but not limited to mutations, amplifications, rearrangements, deletions, and epigenetic gene silencing) in one or more cell-cycle checkpoint regulators which allow the cell to move from one phase of the cell cycle to another unchecked.

One cancer in particular, metastatic castration-resistant prostate cancer (mCRPC), is responsible for the second most cancer fatalities in men in the United States (Siegel R, Naishadham D, Jemal A. Cancer statistics, 2017. CA Cancer J Clin 2012; 62:10-29). One focal point of mCRPC research is the disruption of mitosis in rapidly dividing malignant cells (Mackler N J, Pienta K J, Drug insight: Use of docetaxel in prostate and urothelial cancers. Nat Clin Pract Urol 2005; 2:92-100). The current standard first-line chemotherapy for patients with mCRPC is docetaxsel, which arrests cells in prometaphase by binding to microtubules early in mitosis. It prevents cells from progressing to anaphase by forming multipolar spindles and maintaining activation or the spindle assembly checkpoint (Weaver B A, Cleveland D W. Decoding the links between mitosis, cancer, awl chemotherapy: The mitotic checklist, adaptation, and cell death. Cancer Cell 2005; 8:7-12). Docetaxel causes severe side effects including irreversible neuropathy, as microtubules in non-dividing cells are targeted as well. Furthermore, most tumors exhibit inherent resistance to docetaxel or acquire resistance during treatment

Despite the FDA approval of novel therapies, cabazitaxel, abiraterone acetate and sipuleucel-T, for mCRPC patients, treatment options for this group of patients are limited. Moreover, such approved therapies often cause severe side effects and increase the life span of mCRPC patients by only a few months (Palter C J. Antonarakis E. Sipuleucel-T for the treatment of metastatic prostate cancer: Promise and challenges. Hum Vaccin Immunother 2012; 8, Antonarakis E S, Eisenberger M A. Expanding treatment options for metastatic prostate cancer. N Engl J Med 2011; 364:2055-8). Therefore, there is a need to develop novel therapies that target mCRPC.

Histone deacetylases (HAT) assist in the cell's control of the coiling and uncoiling of DNA around histones, in order to carry out gene expression. The histone deacetylases accomplish this by acetylating the lysine residues in core histones leading to a less compact and more transcriptionally active chromatin. To the converse, the actions of histone deacelytases (HDAC) also remove the acetyl groups from the lysine residues leading to the formation of a condensed and transcriptionally silenced chromatin. Reversible modification of the terminal tails of core histories constitutes the major epigenetic mechanism for remodeling higher-order chromatin structure and controlling gene expression. HDAC inhibitors (HDI) block this action and can result in hyperacetylation of histones, thereby affecting gene expression. Thagalingam S., Cheng K H, Lee H J et al., Ann. N.Y. Acad. Sci. 983: 84-100, 2003; Marks P A. Richon V M, Rifkind R A, J. Natl. Cancer Inst. 92 (15) 1210-1216, 2000; Dokmanovic M, Clarke C., Marks P A, Mol. Cancer Res. 5 (10) 981-989, 2007.

Histone deacetylase (HDAC) inhibitors are a new class, of cytostatic agents that inhibit the proliferation of tumor cells in culture and in vivo by inducing cell cycle arrest, differentiation and/or apoptosis. Historic acetylation and deacetylation play important roles in the modulation of chromatin topology and the regulation of gene transcription. Histone deacetylase inhibitors induces the accumulation of hyperacetylated nucleosome core histones in most regions of chromatin but affects the expression of only a small subset of genes, leading to transcriptional activation of some genes, but repression of an equal or larger number of other genes. Non-histone proteins such as transcription factors are also targets for acetylation with varying functional effects. Acetylation enhances the activity of some transcription factors such as the tumor suppressor p53 and the erythroid differentiation factor GATA-1 but may repress transcriptional activity of others including T cell factor and the co-activator ACTR. Recent studies [. . . ] have shown that the estrogen receptor alpha (ERalpha) can be hyperacetylated in response to histone deacetylase inhibition, suppressing ligand sensistivity and regulating transcriptional activation by histone deacetylase inhibitors. Conservation of the acetylated ERalpha motif in other nuclear receptors suggests that acetylation may play an important regulatory role in diverse nuclear receptor signaling functions. A number of structurally diverse histone deacetylase inhibitors have shown potent antitumor efficacy with little toxicity in vivo in animal models. Several compounds are currently in early phase clinical development as potential treatments for solid and hermatological careers both as monotherapy and in combination with cytotoxics and differentiation agents.” (Vigushin D. M. I; Coombes R. C., http:www.ingentaconnect.com/content/ben/ccdt/2004/0000004/00000002/art00007) (2004)

HDAC inhibitors are classified based on their homology of accessory domains to yeast histone deacetytases. The 18 currency known histone deacetylases are classified into four groups (groups I-IV): Class I, which includes HDAC-1, -2, -3 and -8 are related to yeast RPD3 gene; Class II, which includes HDAC-4, -5, -6, -7, -9 and -10 are related to yeast Hda1 gene; Class III also known as sirtuins are related to the Sir2 gene and include SIRT1-7; and Class IV, which contains only HDAC II and has features of both Class I and Class II. (HDC Inhibitors Database, www.hdacis.com/index/html)

Examples of HDAC inhibitors are as follows. Classical HDIs act exclusively on Class I and Class II HDACs by binding so the zinc-containing catalytic domain of the HDACs. These classical HDIs fall several groupings, in order of decreasing potency:
1. hydroxamic acids (or hydroxamates), such as trichostatin A;
2. cyclic tetrapeptides (such as trapoxin B), and the depsipeptides;
3. benzamides;
4. electrophilic ketones; and
5. the aliphatic acid compounds such as phenylbutyrate and valproic acid. “Second-generation” HDIs include the hydroxamic acids Vorinostat (SAHA), Belinostat (PXD101), LAQ24 (Drummond D C, Noble C O, Kirpotrin D B, Guo Z et al., Annu. Rev. Pharmacol. Toxicol. 45:495-528, 2005), and Panobinostat (LBH589); and the benzamides: Entinostat (MS-27S), C1994, and mocetinostat (MGCD0103) (Beckers T, Burkhardt C et al., Int. J. Cancer 121 (5): 1138-1148, 2005 and Acharya M R, Sparreboom A. et al., Mol. Pharmacol 68 (4): 917-932, 2008). The sirtuin Class III HDACs are dependent on NAD⁺ and are, therefore, inhibited by nicotinamide, as well as derivatives of NAD, dihydrocoumarin, nahpthopyranone, and 2-hydroxynaphaldehydes.

In recent years, there has been an effort so develop HDAC inhibitors as cancer treatments and/or as adjunct therapy. Mark P A. et al. Expert Opinion on Investigational Drugs 14 (12): 1497-1511 (2005) and AACR Journals.org/content/8/3/662, “Histone Deacetylase Inhibitors: A new class of Potential Therapeutic Agents for Cancer Treatment” 2002). The exact mechanisms by which the compounds may work are unclear, but epigenetic pathways have been studied to help elucidate the exact biological pathways. Claude Monneret, Anticancer Drugs 18(4):363-370 2007. For example, HDAC inhibitors have been shown to induce p21 (WAFI) expression, a regulator of p53's tumor suppressor activity. Rochon V M. et al., Proc. Natl. ACad. Sci. U.S.A. 97(18): 10014-10019, 2000. HDACs are involved in the pathway by which the retinoblastoma protein (pRb) suppresses cell proliferation. The pRb protein is part of a complex that attracts HDACs to the chromatin so that it will deacetylate histones. Brehm A. et al., Nature 391 (6667): 597-601, 1998. HDAC1 negatively regulates the cardiovascular transcription factor Kruppel-like factor 5 through direct interaction. Matsumura T. et al., J. Biol. Chem. 280 (13): 12123-12129, 2005. Estrogen is well-established as a mitogenic factor implicated in the tumorigenesis and progression of breast cancer via its binding to the estrogen receptor alpha (ERα). Recent data indicate that chromatin inactivation mediated by HDAC and DNA methylation is a critical component of ERα silencing its human breast cancer cells. Zhang Z. et al., Breast Cancer Res. Treat. 94(1): 11-16, 2005.

Despite all that is believed known and published with respect to the HDAC mechanism of action and inhibition of downstream biological events, it is unknown what any specific anti-cancer therapeutic when used in combination with one or more HDAC inhibitors may afford with respect to being able to improve and/or provide superior treatments for cancer over the standard of care. There is always a need to improve upon the present treatments and/or to provide treatment for cancer to patients who otherwise lose and/or diminish responsiveness to their current treatment.

### BRIEF DESCRIPTION OF THE DRAWINGS/FIGURES

FIGS. 1-a and 1-b depicts the effects on cellular proliferation of prostate cancer cells in-vitro after treatment with AMG 900 and HDACs valproic acid or vorinostat, alone or in combination;

FIG. 2 depicts the effects on expression of phospho-histone H3 is prostate cells in-vitro after treatment with AMG 900 and HDACs valproic acid or vorinostat, alone or in combination;

FIG. 3 is a bar graph depicting the clonogenic survival in prostate cells in-vitro after combination treatment with AMG 900 and HDACs valproic acid or vorinostat;

FIG. 4 is a bar graph depicting the effect on senescence in prostate cells in-vitro after combination treatment with AMG 900 and HDACs valproic acid or vorinostat;

FIG. 5 depicts the effects on expression of p21 in prostate cells in-vitro after treatment with AMD 900 and HDACs valproic acid or vorinostat, alone or in combination;

FIG. 6 is a graph depicting the in-vitro effects on tumor growth after treatment with AMG 900 and HDACs valproic acid or vorinostat, alone or in combination; and

FIG. 7 is a bar graph depicting the in-vivo effects on expression of phospho-histone H3 after treatment with AMG and HDACs valproic acid or vorinostat, alone or in combination; and

FIGS. 8A-8D are bar graphs depicting the clonogenic survival in prostate cells in-vitro after combination treatment with AMG 900 and HDACs valproic acid or vorinostat.

### BRIEF DESCRIPTION OF THE INVENTION

The present invention provides for use of the compound, N-(4-((3-(2-amino-4-pyrimidinyl)-2-pyridinyl)oxy)phenyl)-4-(4-methyl-2-thienyl)-1-phthalazinamine (also referred to herein as “AMG 900” or “the compound”) or a pharmaceutically acceptable salt form thereof, in combination with an HDAC inhibitor for the treatment of cancer. AMG 900 when administered with an HDAC inhibitor exhibited surprisingly superior and synergistic effects above what one of ordinary skill in the art would expect from treatment with either AMG 900 or the HDAC inhibitor alone. More specifically, it was unexpectedly discovered that the combination of AMG 900 in conjunction with an HDAC inhibitor slowed the growth and/or progression of the tumor to the same extent and even greater than the same tumor having been treated with a larger dose of AMG 900 alone. This enhanced efficacy can translate into reduced dosages administered to the cancer patient and possibly shorter dosage regimens. To this end, combination treatments having such synergies not only afford, therefore, an improved treatment for the cancer but may also represent a potential cost savings and improved convenience for the patient. The costs savings can be realized in the form of a lower dosage and/or fewer dosages of AMG 900 being administered to the patient. In addition, the dosage amounts and frequency of the HDAC inhibitor of choice may also be reduced, resulting in as cost savings as well, to both the patient and the insurance company payor on behalf of the patient.

AMG 900 has a chemical structure of:

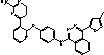 [SMILES: Cc1csc(-c2nnc(Nc3ccc(Oc4ncccc4-c4ccnc(N)n4)cc3)c3ccccc23)c1]

The invention farther provides use of a pharmaceutical composition comprising this compound, or a pharmaceutically acceptable salt form thereof, for therapeutic, prophylactic, acute or chronic treatment of cancer, including solid tumors of prostate cancer, breast cancer, ovarian cancer, lung cancer and the like, and other proliferating cancerous cells in patients. In one embodiment, the invention provides the use of AMG 900 in the manufacture of medicaments, of pharmaceutical compositions, for the treatment of cancer in subjects who are in need of treatment, or who may have been previously treated solely with an HDAC inhibitor. The invention further contemplates using AMG 900 in combination with such HDAC inhibitors presently approved for medical use by regulatory agencies, including without limitation Vorinostat and Romidepsin, as well as other HDAC agents undergoing clinical trials, including without limitation Panobinostat (LBH589), Valproic acid (as Mg valproate); Belinostat (PXD101), Mocetinostat (MGCD103), Abexinostat (PCI-24781), Entinostat (MS-275), SB939, Resminostat (4SC-210), Givinostat (ITF2357), CUDC-101, AR-42, CHR-2845, CHR-3996, 4SC-202, CG200745, ACY-1215 and Sulforaphane. The invention also includes use of any other pre-clinical HDAC inhibitors determined useful for the treatment of cancer via the reduction or inhibition of histone deacetylase activity in combination with AMG 900 for the treatment of cancer. In another embodiment, the invention provides a method of treating a solid tumor, including non-small cell lung cancer, breast cancer, and prostate cancer in a subject, the method comprising administering to the subject an effective dosage amount of AMG 900 or a pharmaceutically acceptable salt thereof, in combination with an HDAC inhibitor to treat the solid tumor.

### DETAILED DESCRIPTION OF THE INVENTION

AMG 900, a small molecule pan Aurora A, B and C kinase inhibitor, has been found to provide a surprising and unexpected advantage its effect when use or administered in combination with a histone deacetylase (HDAC) inhibitor agent. Particularly, AMG 900 when administered, in dosages lower than when administered alone, in combination with as HDAC inhibitor exhibited positive and synergistic anti-proliferative effects and long term clonogenic survival effects on cancer cells. Further, such combination treatments of tumors in-vivo exhibited effects on the growth curve of the tumor at least equivalent to, and superior than, that which was exhibited with a higher dosage of AMG 900 alone. Finally, the effects of the combination treatment were moderately synergistic to strongly synergistic compared to the single agent use, depending upon the concentrations used.

### DEFINITIONS

The following definitions should further assist in understanding the scope of the invention described herein.

The terms “cancer” and “cancerous” when used herein refer to or describe the physiological condition in subjects that is typically characterized by unregulated cell growth. Examples of cancer include, without limitation, carcinoma, lymphoma, sarcoma, blastoma and leukemia. More particular examples of such cancers include squamous cell carcinoma, lung cancer, pancreatic cancer, cervical cancer, bladder cancer, hepatoma, breast cancer, colon carcinoma, and head and neck cancer. While the term “cancer” as used herein is not limited to any one specific form of the disease, it is believed that the combination treatment methods provided by the invention will be particularly effective for is variety of cancers in a subject in need of treatment.

The term “HDAC inhibiting agent” when used herein refers to a histone deacetylase inhibiting agent, which alone, may be useful for treating cancer. This term additionally refers to antineoplastic drugs used to treat cancer or a combination of these drugs into a standardized treatment regimen. Examples of HDAC inhibiting agents include, without limitation, member agents of Classes I-IV. For instance, examples of HDAC inhibiting agents include, without limitation, those which are approved for human use such as Vorinostat and Romidepsin, those which have been regulatory approved for and are undergoing human clinical trials such as Panobinostat (LBH589; Ph III for various cancers including cutaneous T-cell lymphoma), valproic acid (administered as Mg valproate salt; in Ph III trials for cervical and ovarian cancer); Belinostat (PXD101; Ph II trial for relapsed ovarian cancer, and reportedly good results for T cell lymphoma), Mocetinostat (MGCD103; undergoing Ph II clinical trials for various cancers, including follicular lymphoma, Hodgkin lymphoma and acute myeloid leukemia (AML)), Abexinostat (PCI-24781; undergoing Ph II clinical trial, for sarcoma and lymphoma), Entinostat (MS-275; undergoing clinical trials Hodgkin lymphoma, lung cancer and breast cancer), SB939 (In Ph II for recurrent or metastic prostate cancer (HRPC), and has shown good Ph II results for myelofibrosis), Resminostat (4SC-201; exhibited good results for Hodgkin lymphoma, and has met a primary endpoint in a Ph II trial for hepatocellular carcinoma), Givinostat (ITF2357; undergoing clinical trials for refractory leukemias and lyelomas), CUDC-101 (in Ph I for safety and intended for Ph II cancer trials), AR-42 (started clinical trials for various cancers including relapsed or treatment resistant multiple myeloma, chronic lymphmocytic leukemia or lymphoma), CHR-2845, CHR-3996, 4SC-202 (for selective hematological indications), CG200745 (in clinical trails for solid tumors), ACY-1215 (in clinical trials for multiple myeloma), and Sulforaphane, a novel histone deacetylase inhibitor in clinical trials as an anti-cancer agent. The invention also includes use of any other pre-clinical HDAC inhibitors, including without limitation, Kevetrin, an agent selective for HDAC2, that are later determined to be useful for the treatment of cancer via the reduction or inhibition of histone deacetylase activity in combination with AMG 900.

The term “comprising” is meant to be open ended, including the indicated component(s) but not excluding other elements.

The term “refractory” when used here is intended to refer to not-yielding to, resistant or non-responsive to treatment, stimuli (therapy) or cure, including resistance to multiple therapeutic curative agents. “Refectory” when used herein in the context of characterizing a cancer or tumor is intended to refer to the cancer or tumor being non-responsive or having a resistant or diminished response to treatment with one or more anticancer agents. The treatment typically is continual, prolonged and/or repetitive over a period of time resulting in the cancer or tumor developing resistance or becoming refractory to that very same treatment.

The term “subject” as used herein refers to any mammal, including humans and animals, such as cows, horses, dogs had eats. Thus, the invention may be used in human patients as well as in veterinarian subjects and patients. In one embodiment of the invention, the subject is a human.

The phrase “effective dosage amount” or “therapeutically-effective” when used in conjunction with AMG 900 or an HDAC inhibiting agent, is intended to quantify the amount of the compound (AMG 900 or HDAC inhibiting agent), which will achieve a reduction in size or severity of the cancer or tumor.

The terms “treat”, “treating” and “treatment” as used herein refer to therapy, including without limitation, curative therapy, prophylactic therapy, and preventative therapy. Prophylactic treatment generally constitutes either preventing the onset of disorders altogether or delaying the onset of a pre-clinically evident stage of disorders in individuals.

The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of the compound may be prepared from an inorganic acid or from an organic acid. Examples of such inorganic acids include, without limitation, hydrochloric, hydrobromic, hydroiodic, nitric, carbonic, sulfuric and phosphoric acid. Examples of organic acids include, without limitation, aliphatic, cycloaliphatic, aromatic, arylaliphatic, heterocyclic, carboxylic and sulfonic classes of organic acids, examples of which are formic, acetic, adipic, butyric, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic phenylacetic, mandelic, embonic (pamoic), methanesulfonic, ethanesulfonic, ethanedisulfonic, benzensulfonic, pantothenic, 2-hydroxyethanesulfonic, toluenesulfonic, sulfanilic, cyclohexylaminosulfonic, camphoric, amphorosulfonic, digluconic, cyclopentanepropionic, dodecylsulfonic, glucoheptanoic, glycerphosphonic, heptanoic, hexanoic, 2-hydroxy-ethanesulfonic, nicotinic, 2-naphthalenesulfonic, oxalic, palmoic, pectinic, persulfuric 2-phenylpriopionic, picrie, pivalic propionic, succinic, tartaric, thiocyanic, mesylic, undecanoic, stearic, algenic, β-hydroxybutyric, salicylic, galactaric and galacturonic acid. It is also within the scope of the invention herein to have multiple counter ion salts associate with the free base of AMG 900 and/or the HDAC inhibiting agent. For instance, and without limiting the scope of the invention, a bis-HCl, bis-sulfonic acid, bis-methanesulfonic acid, bis-benzenesulfonic acid, bis-toluenesulfonic acid, bis-aspartic acid, bis-malic acid or a bis-glutamic acid salt of AMG 900 is contemplated herein to be useful in combination with an HDAC inhibiting agent to treat cancer.

Suitable pharmaceutically-acceptable base addition salts of the compound include, without limitation, metallic salts such as salts made than aluminum, calcium, lithium, magnesium, potassium, sodium and zinc, or salts made from organic bases including primary, secondary, tertiary amines and substituted amines including cyclic amines such as caffeine, arginine, diethylamine, N-ethyl piperidine, aistidine, glucamine, isopropylamine, lysine, morpholine, N-ethyl morpholine, piperazine, piperidine, triethylamine, trimethylamine. All of the salts contemplated herein may be prepared by conventional means from the corresponding compound by reacting, for example, the appropriate acid or base with the compound. A base addition salt of a carboxylic acid or similar functional group on the HDAC inhibiting agent administered may be useful in carrying out the invention described herein. Some useful HDAC inhibiting agents which may readily form base addition salts include, without limitation, hydroxamic acids or hydroxamates such as trichostatin A, vorinostat (also referred to herein as SAHA), belinostat and panobinostat, and aliphatic acid compounds such as phenyl butyrate and valproic acid.

AMG 900, N-(4-((3 -(2-amino-4-pyrimidinyl)-2-pyridinyl)oxy)phenyl)-4-(4-methyl-2-thienyl)-1-phthalazinamine, may be prepared by the procedure analogous to that described in PCT publication WO2007087276, Example Methods A1 or A2 mm on pg 70 but using 1-chloro-4-(4-methyl-2-thienyl)phthalazine as the starting material in conjunction with Examples 15 (pg 50), 25 (pg 55) and 30 (pg 59). These procedures are also described in U.S. Pat. No. 7,560,551, which specification is hereby incorporated herein by reference in its entirety. Specifically, AMG 900 maybe prepared as described in Example 1 below.

### Example 1

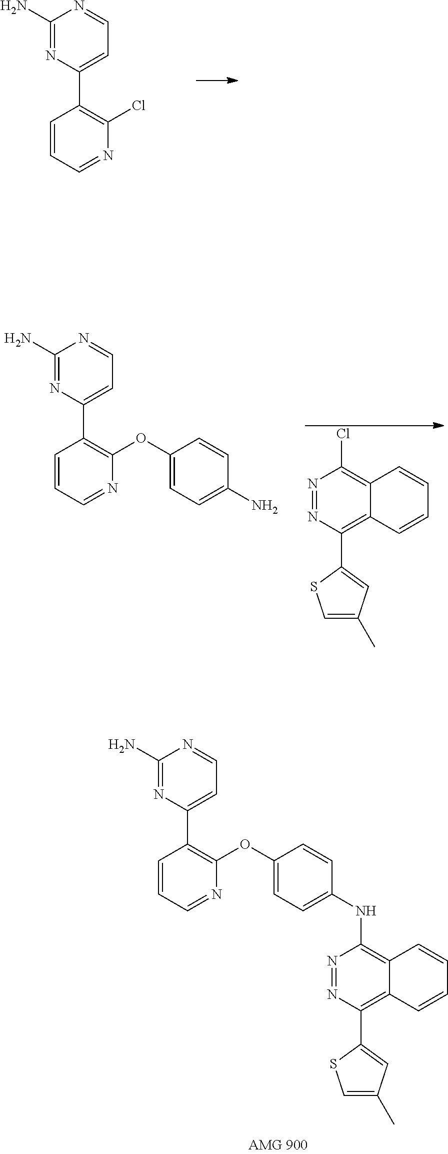 [SMILES: Nc1nccc(-c2cccnc2Cl)n1 ; Nc1ccc(Oc2ncccc2-c2ccnc(N)n2)cc1 ; Cc1csc(-c2nnc(Cl)c3ccccc23)c1 ; Cc1csc(-c2nnc(Nc3ccc(Oc4ncccc4-c4ccnc(N)n4)cc3)c3ccccc23)c1]

### Synthesis of N-(4-((3-(2-amino-4-pyrimidinyl)-2-pyridinyl)oxy)phenyl)-4-(4-methyl-2thienyl)-1-phthalazinamine (AMG 900)

Step 1: 4-(2-chloropyridin-3-yl)pyrimidin-2-amine

In an argon purged 500 mL round bottom flask placed in an isopropanol bath, was added sodium metal (3.40 g, 148 mmol) slowly to methanol (180 mL). The mixture was stirred at room temperature (RT) for about 30 minutes. To this was added guanidine hydrochloride (12.0 mL, 182 mmol) and the mixture was stirred at RT for 30 minutes, followed by addition of (E)-1-(2-chloropyridin-3-yl)-3-(dimethylamino)prop-2-en-1-one (12.0 g, 57.0 mmol), attached air condenser, moved reaction to an oil bath, where it heated to about 50° C. for 24 h. Approximately half of the methanol was evaporated under reduced pressure and the solids were filtered under vacuum, then washed with saturated sodium bicarbonate (NaHCO₃), and H₂O, air dried to yield 4-(2-chloropyridin-3-yl)pyrimidin-2-amine as off white solid. MS m/z=207 [M+1]⁺. Cal'd for C₉H₇ClN₄: 206.63.

Step 2: 4-(2-(4-aminophenoxy)pyridin-3-yl)pyrimidin-2-amine

To a resealable tube was added 4-aminophenol (1.3 g, 12 mmol), cesium carbonate (7.8 g, 24 mmol), and DMSO (16 ml, 0.75 M). The mixture was heated to 100° C. for 5 minutes, and then 4-(2-chloropyridin-3-yl)pyrimidin-2-amine (2.5 g, 12 mmol) was added, and the reaction mixture was heated to 130° C. overnight. Upon completion, as judged by LCMS, the reaction mixture was allowed to cool to RT and diluted with water. The resulting precipitate was filtered, and the solid washed with water and diethyl ether. The solid was then taken up in 9:1 CH₂Cl₂:MeOH and passed through a pad of silica gel with 9:1 CH₂Cl₂:MeOH as eluent. The solvent was concentrated in vacuo to provide the desired product, 4-(2-(4-aminophenoxy)pyridin-3-yl)pyrimidin-2-amine. MS m/z=280 [M+1]⁺. Calc'd for C₁₅H₁₃N₅O: 279.30.

Step 3: 1-Chloro-4-(4-methylthiophen-2-yl)phthalazine

1,4 Dichlorophthalazine (1.40 g, 7.03 mmol), 4-methylthiophen-2-ylboronic acid (999 mg, 7.03 mmol), and PdCl₂(DPPF) (721 mg, 985 μmol) were added into a sealed tube. The tube was purged with Argon. Then sodium carbonate (2.0 M in water) (7.74, ml, 15.5 mmol) and 1,4-dioxane (35.2 ml, 7.03 mmol) were added. The tube was sealed, stirred at RT for 5 min, and placed in a preheated oil bath at 110° C. After 1 h, LC-MS showed product and byproduct (double coupling), and starting material dichlorophthalazine. The reaction was cooled to RT, filtered through a pad of celite with an aid of ethyl acetate (EtOAc), concentrated, and loaded onto column. The product was purified by column chromatography using Hex to remove the top spot, then 80:20 hexanes:EtOAc to collect the product. The product, 1-chloro-4-(4-methylthiophen-2-yl)phthalazine was obtained as yellow solid. LC-MS showed that the product was contaminated with a small amount of dichlorophthalazine and biscoupling byproduct. MS m/z=261 [M+1]⁺. Calcd for C₁₃H₉ClN₂S: 260.12.

Step 4: N-(4-((3-(2-amino-4-pyrimidinyl)-2-pyridinyl)oxy)phenyl)-4-(4-methyl-2-thienyl)-1-phthalazinamine

To 4-(2-(4-aminophenoxy)pyridin-3-yl)-pyrimidin-2-amine and 1-chloro-4-(4-methyl-2-thienyl)phthalazine was added tBuOH. The resulting mixture was heated at 100° C. in a sealed tube for 16 hours. The reaction was diluted with diethyl ether and saturated sodium carbonate and vigorously shaken. The resulting solids were filtered and washed with water, diethyl ether and air dried to yield N-(4-((3-(2-amino-4-pyrimidinyl)-2-pyridinyl)oxy)phenyl)-4-(4-methyl-2-thienyl)-1-phthalazinamine as an off-white solid. MS m/z=504 [M+H]⁺. Calc'd for C₂₈H₂₁N₇O S: 503.58.

### LC-MS Method:

Samples were run on a Agilent model-1100 LC-MSD system with an Agilent Technologies XDB-C₈ (3.5μ) reverse phase column (4.6×75 mm) at 30° C. The flow rate was constant and ranged from about 0.75 mL/min to about 1.0 mL/min.

The mobile phase used a mixture of solvent A (H₂O/0.1% HOAc) and solvent B (AcCN/0.1% HOAc) with a 9 min time period for a gradient from 10% to 90% solvent B. The gradient was followed by a 0.5 min period to return to 10% solvent B and a 2.5 min 10% solvent B re-equilibration (flush) of the column.

Other methods may also be used to synthesize AMG 900. Many synthetic chemistry transformations, as well as protecting group methodologies, useful in synthesizing AMG 900, are known in the art. Useful organic chemical transformation literature includes, for example, R. Larock, Comprehensive Organic Transformations, VCH Publishers (1989); T. W. Greene and P. G. M. Wuts, Protective Groups in Organic Synthesis, 3^{rd} edition, John Wiley and Sons (1999); L. Fieser and M. Fieser, Fieser and Fieser's Reagents for Organic Synthesis, John Wiley and Sons (1994); A. Katritzky and A. Pozharski, Handbook of Heterocyclic Chemistry, 2^{nd} edition (2001); M. Bodanszky, A. Bodanszky, Use Practice of Peptide Synthesis, Springer-Verlag, Berlin, Heidelberg (1984); J. Seyden-Penne, Reductions by the Alumino- and Borohydrides in Organic Synthesis, 2^{nd} edition, Wiley-VCH, (1997); and L. Paquette, editor, Encyclopedia of Reagents for Organic Synthesis, John Wiley and Sons (1995).

AMG 900 was tested for its ability to reduce or inhibit tumor progression in various cell lines (in-vitro) and multiple solid tumor types (in-vivo), some of which have previously been exposed to and developed resistance to standard-of-care antimitotic agents, including taxanes and vinca alkaloids, as well as to other chemotherapeutic agents. The following Examples and resulting will illustrate the synergistic ability of AMG 900, when used in combination with an HDAC inhibiting agent, to treat cancer. Unless otherwise indicated, the free base form of AMG 900 was used in the Examples described hereinbelow.

### Example 2

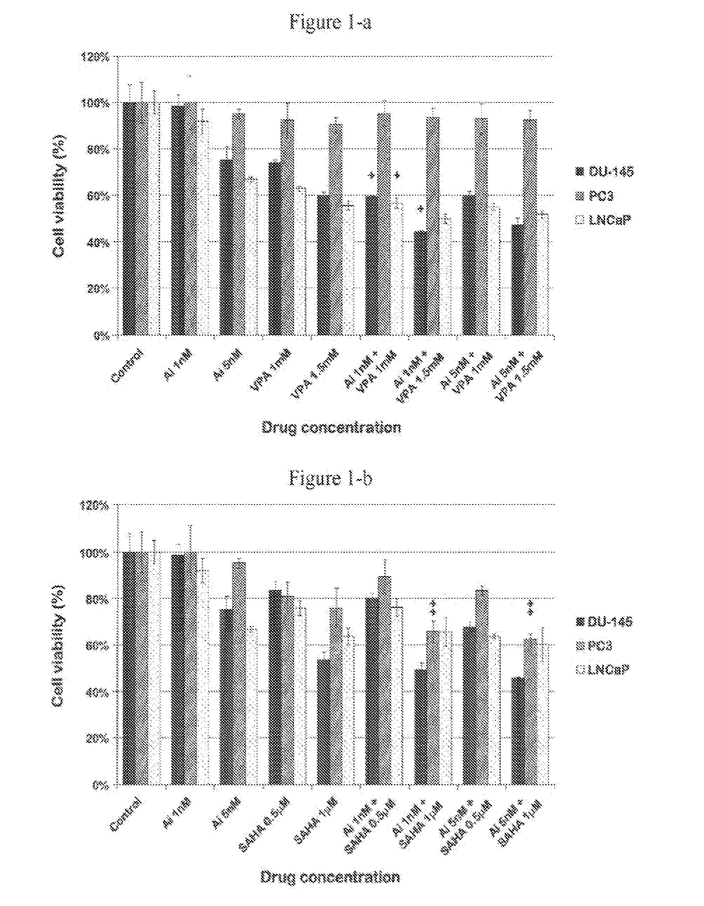

To investigate whether AMG 900-induced suppression of aurora kinase A and B activity inhibits cell proliferation in combination with an HDAC inhibiting agent such as VPA or SAHA, the antiproliferative, expression of phosphor-histone H3 (pH3) and/or clonogenic survival effect of AMG 900 and as HDAC inhibiting agent, alone and in combination, were evaluated in vitro using prostate cancer cell lines. As shown in FIGS. 1-a, 1-b, 2,3, 4 and 5, AMG 900 exhibited useful and synergistic antiproliferative, cell survival and reduced downstream expression activity in prostate cancer cells. This anti cancer activity was seen with low concentrations and dosages of AMG 900. Importantly, lower dosages of AMG 900, in combination with an HDAC inhibiting agent, such as VPA or Vorinostat, provide the same or greater effect on cellular apoptosis and, therefore at treating cancer, than with a higher dose of AMG 900 as a single agent

As shown in FIGS. 1-a and 1-b, proliferation activity of prostate cancer cells, DU-145, PCS and LNCaP cell lines, decreased after combination treatment with AMG 900 and HDACs VPA and vorinostat in a moderately synergistic manner at low concentrations in these cells. Further, when AMG 900 was administered in combination with SAHA, it enhanced inhibition of cellular proliferation compared to single agent, use in all three cell lines. Finally, AMG 900 and SAHA treatment combination exhibited synergistic effects in the PC3 cell lines, as illustrated in FIGS 1-a and 1-b.

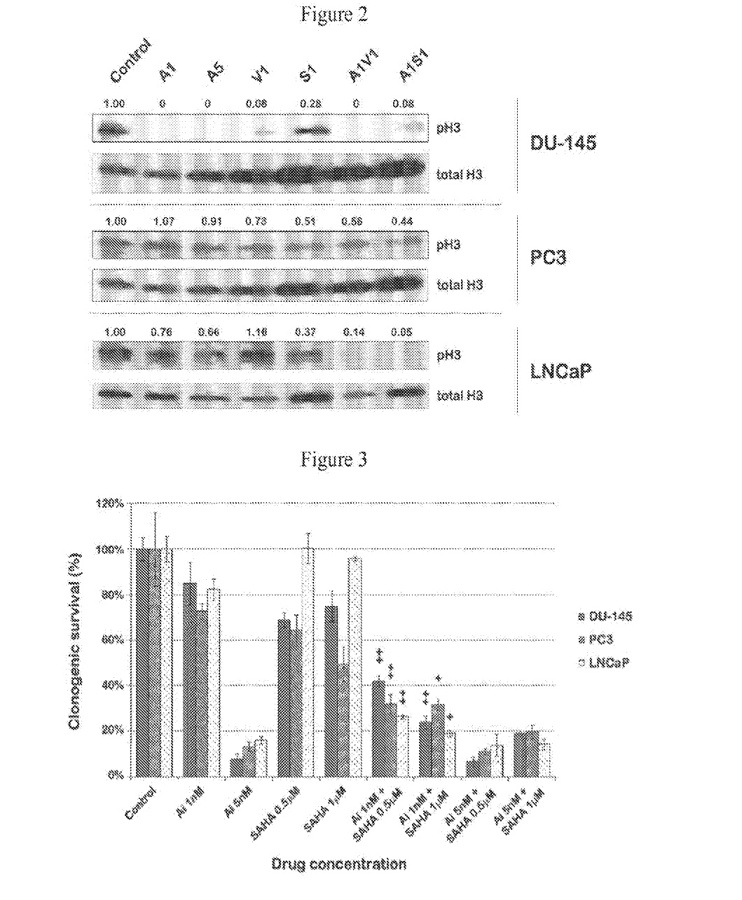

FIG. 2 departs the effects of the treatment of AMG 900 and HDACIs (HDAC inhibitors) valproic acid or vorinostat, alone or in combination, on the expression of phospho-histone H3 in prostate cells. Phosphorylated histone H3 (pH3), an indicate for aurora B activity and regarded as a biological marker for aurora kinase activity generally, is decreased both aster AMG 900 and HDACIs treatment. In PC3 and LNCaP cells protein expression levels of pH3 are more decreased after combination treatment compared to single agent use.

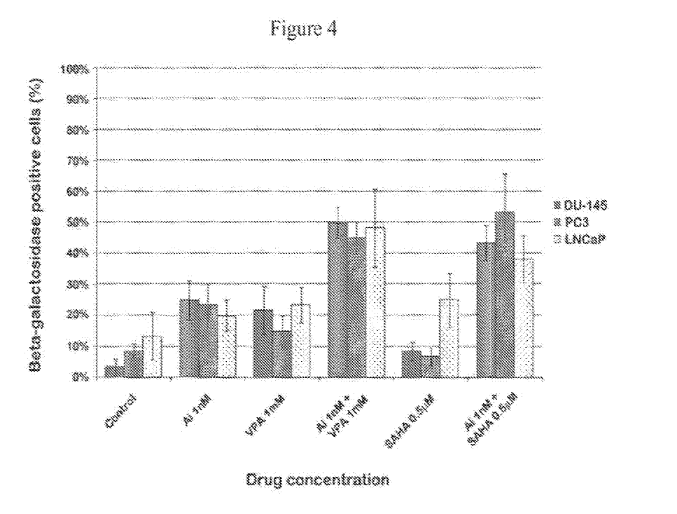

FIG. 3 is a bar graph which depicts the effects of the treatment of AMG 900 and HDACIs valproic acid or vorinostat, alone or in combination, on the clonogenic survival in prostate cells. Clonogenic assays indicate decreased clonogenic survival after combination treatment of low dose AMG 900 with VPA or SAHA. Depending on the concentrations used, the effect was moderately synergistic to strongly synergistic compared to single agent use.

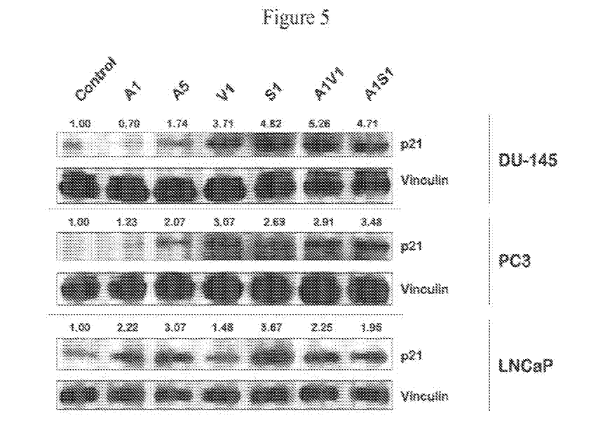

FIG. 4 is a bar graph which depicts the effect of the treatment of AMG 900 and HDACIs valproic acid or vorinostat, alone or in combination, on senescence in prostate cells. Ai represents AMG 900 alone at a 1 nM dose. The HDACI VPA represents valproic acid alone while SAHA represents treatment with vorinostat alone. The concentrations are also provided in the figure. The precent positive cells in SA β-galactosidase assay indicates increased senescence after 48 hours of combination treatment of low dose AMG 900 with VPA or SAHA when compared to single agent use (p<0.05), except for combination treatment of AMG 900 with SAHA its LNCaP cells. Imaging of the PC3 cells during the assay further confirmed this finding.

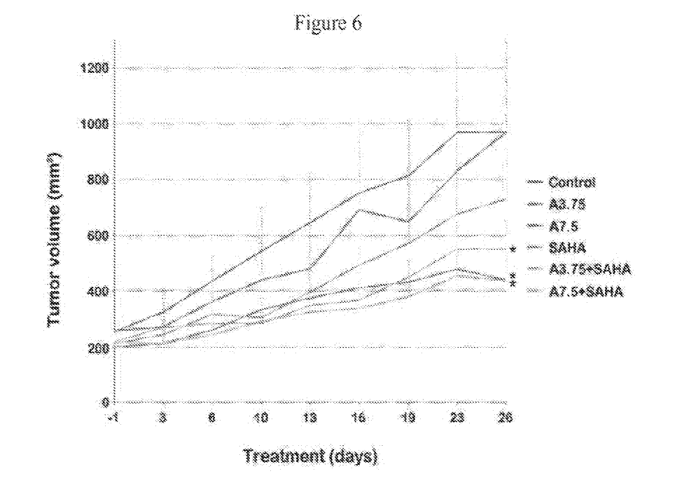

FIG. 5 depicts the effect of the treatment of AMG 900 and HDACIs valproic acid or vorinostat, alone or in combination, on expression of p21 in prostate cells. A1 represents AMG 900 at a low dose, while A5 represents AMG 900 at a higher dose. V1 represents VPA alone while S1 represents treatment with SAHA alone. As seen, p21, a cyclin-dependent kinase inhibitor of cyclin-CDK2 and -4 complexes, increased in each cell line after combined treatment with AMG 900 and HDACIs. This may indicate increased arrest is G1-phase, or simply increased stress in the cells at 48 hours.

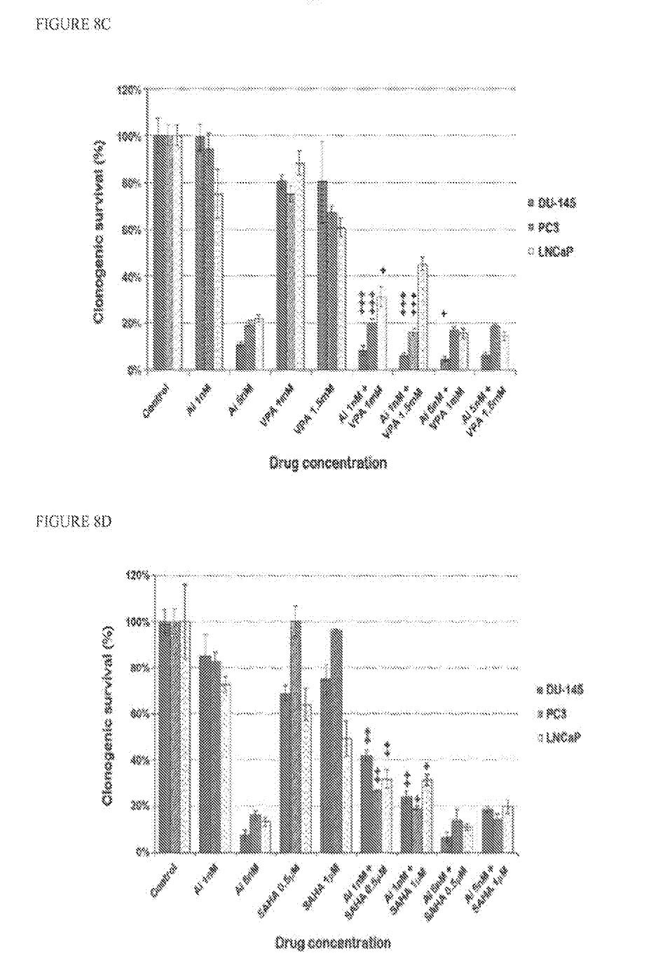

FIGS. 8A through 8D, similar to FIGS. 1, 1-a and 3, are bar graphs depicting the effects of the treatment of AMG 900 and HDACIs valproic acid or vorinostat, alone or in combination, on the proliferation activity and long-term clonogenic survival in prostate cancer cells. The graphs reveal additive and/or synergistic reduction in both proliferation activity and long term clonogenic survival after 48 hours of combination treatment of AMG 900 with VPA or SAHA. Synergy was assessed using calcuSyn. Ai is AMG 900; + represents moderate synergy; ++ represents good synergy; and +++ represents strong synergy. Depending on the concentrations used, the effect was moderately synergistic to strongly synergistic compared to single agent use.

### MATERIALS AND METHODS

Test Materials

| | |
|---|---|
| Test article: | AMG 900 |
| Formulation: | DMSO |
| Source: | Amgen Inc. |
| Test article | Valproic acid |
| | (VPA) |
| Source | Commercially |
| | available - |
| | Sigma Aldrich |
| Test article | Vorinostat |
| | (SAHA) |
| Source | Commercially |
| | available - |
| | AtonPharma |

### In Vitro Methods

### Cell Culture and Treatment

Prostate cancer cell lines (DU-145, LNCaP, PC3) were obtained from ATCC. Cells were grown RPMI-1640 (Invitrogen) with 10% Fetal Bovine Serum (FBS) (Gemini) nd maintained in a 37° C. humidified incubator supplemented with 5% CO₂. Sodium salt of VPA (Sigma-Aldrich) was prepared in RPMI at a IM stock on the day of treatment of the cells. Vorinostat (SAHA) (AtonPharma) and AMG 900 (Amgen) were maintained in 10 mM DMSO stock solutions at −20° C. and diluted in RPMI upon use. For combination studies, compounds were administered concomitantly.

### Cell Viability

MTS assays were performed with CellTiter 96™ Aqueous Non-Radioactive Cell Proliferation Assay reagent (Promega) according to the manufacturer's instructions. In brief, prostrate cancer cells were plated in 96-well plates, allowed to adhere overnight and treated with the compounds for 72 hours. Subsequently, the MTS reagent was added and after about two hours absorption at 490 nm was measured using a colorimetric plate reader (Molecular Devices). Synergy was assessed using CalcuSyn software (BioSoft).

### Cell Survival

Clonogenic assays were performed to assess long term cell survival. Prostate cancer cells were plated in complete RFMI media in 60 mm petri dishes. Upon reaching 50-60% confluency, drugs were added at the appropriate concentration and dishes were incubated for 48 hours. Then cells (1.25×10³ for DU-145 and PC3 cells, 2×10³ for LNCaP cells) were replated and grown in triplicate in new 60 mm dishes containing fresh, complete RPMI media. After 10-14 days, a crystal violet stain (Sigma) was administered to visualize colonies. Viable colonies were counted, and subsequently the presence and degree of synergistic effects were measured using CalcuSyn (Biosoft).

### Cell Senescence

Prostate cancer cells were plated in 6-well plates (25-50×10³ per well) and allowed to adhere overnight. Compounds were added at appropriate concentrations for 48 hours, after which senescent cells were stained using the senescence β-galactosidase staining kit (Cell Signaling Technology) according to the manufacturer's instructions. In brief, cells were washed in PBS and fixed in Fixative Solution (2% formaldehyde and 0.2% glutaraldehyde in 1×PBS). After fixing, cells were washed in PBS and incubated with Staining Solution (containing 40 mM citric acid/sodium phosphate (pH 6.0), 150 mM NaCl, 2 nM MgCl₂, 5 nM potassium ferrocyanide, potassium ferricyanide, and 1 mg/ml X-gal (from a stock solution of 20 mg/ml X-gal in DMF)) in a 37° C. incubator for 24 hours. Cells were washed in PBS and visualized under an Olympus IX70 inverted microscope using an Uplan FL 10×phase contrast lens. Multiple images were taken from each well. Senescent (blue) and total cells were counted in three fields of approximately 30 cells for each treatment.

### Immunoblotting

Western blotting was performed as described previously, Kachhap S K, Rosmus N, et al. “Downregulation of homoglous recombination DNA repair genes by HDAC inhibition in prostate cancer is mediated through the E2F1 transcription factor”, PLoS One 2010; 5:e11208. Loading volumes equalized to 20 μg of total protein were used. Primary antibodies were diluted 1:1000 in blocking solution with the exceptions of vinculin and phosphorylated aurora A-C, which were diluted 1:4000 and 1:500, respectively. Conjugated secondary antibodies were diluted 1:400 in blocking solution. For blocking solution 5% milk is TBST (100 mM Tris-HCl ph 7.4, 0.1% Tween20, 1.50 mM NaCl in H₂O) was used for p21, cyclin B1, vinculin, cleaved PARP and all secondary antibodies; 5% BSA in TBST was used for the primary antibodies phosphorylated aurora, and phosphorylated and total H3. Blots were developed on film and scanned into a computer at 300 dots per inch (dpi). Densitometric analyses were performed using ImageJ (Research Services Branch, National Institute of Mental Health).

### Statistical Analyses and Synergy

Synergysm was determined using the CalcuSyn program (BioSoft), CalcuSyn calculates a combination index (CI) at different levels of growth, using the formula for mutually nonexclusive mechanisms: (D1/Dx1+(D2/Dx2)+D1D2Dx1Dx2), where D1 and D2 are the doses of drug 1 and drug 2 in combination required its produce x percentage effect, and Dx1 and Dx2 are the doses of drug 1 and drug 2 alone required to produce the same effect. Synergism levels (no synergism (CI>0.9), moderate synergism (0.7<CI<0.9, +), synergism (0.3<Cl<0.7, ++), strong synergism (0.1<CI<0.3, +++), very strong synergism (CI<0.1, ++++)) were determined from CI ranges, using the Chou-Talalay method according to the manufacturer's instructions. Chou T C. “Drug Combination Studies and their Synergy Quantification Using the Chou-Talalay Method”, Cancer Res. 70:440-6, 2010. After counting the number of senescent versus non-senescent cells, student's t-tests were performed to determine whether there was a statistically significant difference in the percentage of senescent cells between treatment groups.

### Example 3

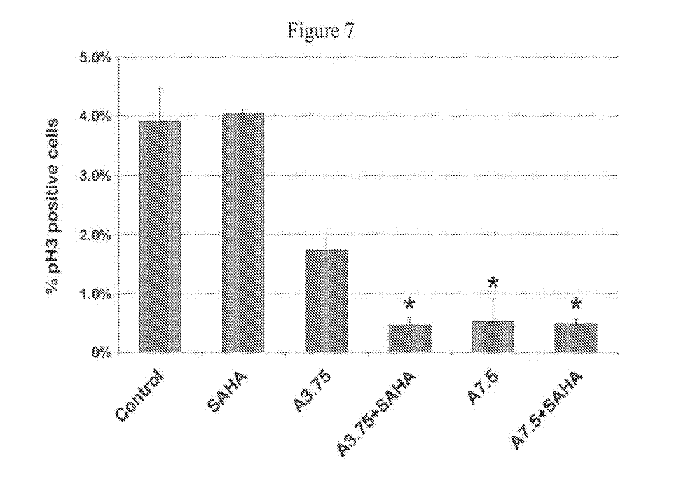

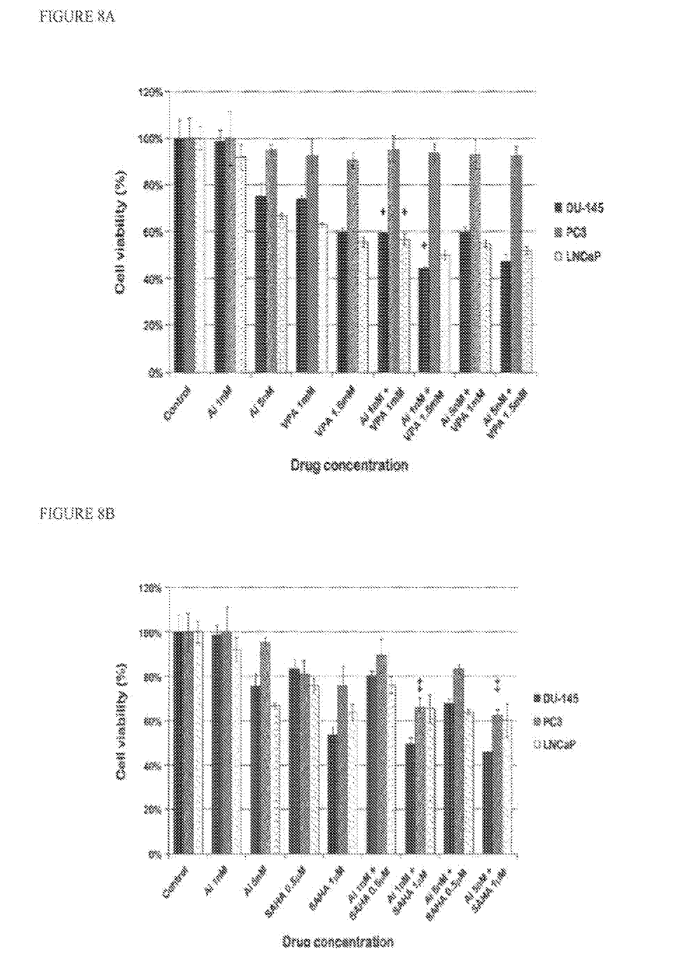

To investigate whether AMG 900 induced suppression of aurora kinase A and B activity inhibits cell proliferation in combination with an HDAC inhibiting agent such as VPA or SAHA, the effect of AMG 900 and an HDAC inhibiting agent alone and in combination, were evaluated in vivo in rodents. As shown in FIGS. 6 and 7, AMG 900 exhibited useful and synergistic effects on tumor growth and reduced downstream expression of phospho-histone H3 (pH3). This anti cancer activity was seen with low concentrations and dosages of AMG 900. Importantly, lower dosages of AMG 900, in combination with an HDAC inhibiting agent, such as VPA or Vorinostat, provide the same or greater effect on tumor growth and, therefore at treating cancer, than with a higher dose of AMG 900 as a single agent.

FIG. 6 is a graph depicting the in-vivo effects on tumor growth after treatment with AMG 900 and HDACIs valproic acid or vorinostat, alone or in combination. A3.75 alone represents AMG 900 at a dosage of 3.75 mg/kg weight of the subject, while A7.5 alone represents AMG 900 at a dosage of 7.5 mg/kg weight of the subject. SAHA alone represents treatment with vorinostat alone. The combined treatment are so indicated by the plus (+) sign. As illustrated, the tumor growth rate of mice treated with low dose AMG 900 and SAHA in combination is significantly decreased compared to the control and low dose single agent treatment (p<0.02), but similar to high dose single agent AMG 900 and high dose combination treatment (p=0.721).

FIG. 7 is a bar graph depicting the in vivo effects on expression of phospho-histone H3 after treatment with AMG 900 and HDACIs valproic acid or vorinostat, alone or in combination.

### In Vivo Methods

### Animals

The animal protocol was approved by the Animal Care and Use Committee of Johns Hopkins University. All institutional Animal Care and Use Committee guidelines and United States Department of Agriculture regulations were followed. The mice used in this study were 8-week old JHU Oncology NOD/SCIDs and were housed under aseptic conditions on a 12 hour light-dark cycle with food and water provided ad lib. Each cage contained ≦5 mice; cages were differentiated by treatment groups.

Two million DU-145 prostate cancer cells, immersed in complete RPMI media, were embedded in a 1:2 solution of matrigel, and injected subcutaneously into the right flank of the mice. The tumor inoculation success rate was approximately 90%. Following a 3 week growth incubation period, tumor volume was estimated its mm³ by measurement of tumor dimensions with digital calipers, using the standard formula: length*width*height. Before treatment initiation, mice were divided into homogenous groups (8-9 per group) according to tumor size. Once tumors reached a volume of 100-400 mm³, mice began receiving medications in accordance with their assigned treatment group. Medications were administered in four-day cycles per week for a total duration of four weeks. SAHA (50 mg/kg) was administered once daily via intraperitoneal injections, on mornings of days 1-4 of the dosing cycle, AMG 900 was administered through gavage in either low or high doses, depending on the treatment group, on days 1 and 2 of each dosing cycle. Mice were treated with vehicle (2% HPMC (hydroxylpropyl methyl cellulose) and 1% Tween80 in DI (deionized) water, 2.2 with MSA (methane sulfonic acid)), or AMG 900 at a concentration of 3.75 mg/kg or 7.5 mg/kg (provided weekly by AMGEN). Tumor measurements and mice bodyweights were obtained on the day preceding each dosing cycle (day 0/7), as well as the final day of dosing (day 4). Tumors were collected 26-35 days after start of the treatment, when tumor volume reached 100 mm³. For histological assessment of the tumors, mice were cardiac perfused with 2% paraformaldehyde, after which tumors were excised, infiltrated in sucrose, embedded in OCT (Optimal Cutting Temperature) media and stored at −80° C. Tissue sections were prepared for hematoxylin-eosin staining by fixing the tissue in formalin and embedding it in paraffin after excision of the tumor.

### Tissue Immunostating

Sections (20 μm) were cut from frozen tissues mounted in OCT (Sakura Finetek) and mounted on microscopic slides (Fisher Scientific). Sections were blocked in blocking buffer (5% goat serum, 0.5% BSA, 0.1% TritonX 100 and 0.01% azide in PBS) and probed overnight with the primary antibody phosphorylated histone H3 in a 1:100 dilution (cell signaling technology). Then the sections were washed in PBS containing 0.1% Triton-X 100 and probed overnight with Alexa-fluor conjugated secondary antibody anti-Rabbit 546 in a 1:500 dilation (Molecular Probes, Invitrogen). Subsequently sections were washed in PBS and fixed with 10% formalin, and the nuclei were stained with Prolong Gold with DAPI (Molecular Probes, Invitrogen). Coverslips were mounted on the slides and the sections were imaged with a Nikon Eclipse Ti microscope 20× (Nikon). The percentage of fluorescent cells was assessed in at least three fields of view per treatment group by dividing the density of red (phosphorylated, histone H3 positive cells) by the density of blue (DAPI staining all nuclei) with ImageJ.

### Statistical Methods

The in vivo data from the DU-145 xenograft model was analyzed with a random intercept hierarchical linear model. The primary statistical outcome was tumor volume. To adjust for the initial volume, for each mouse, tumor volumes on days 3 through 26 were divided by the volume on day −1 and then the log of these values was taken for analysis. The intercept in this longitudinal model was specified such that it represented the log ratio of the final tumor volume to the initial volume (time was coded using negative numbers and 0 the final day). The model that was fit had the form:

γᵢₜ=β₀+ζ₀₁+β₁ timeᵢₜ+β₂ groupᵢ+β₃ (groupᵢ*timeᵢₜ)+εᵢₜ

where γᵢₜ denotes the log ratio tumor volume for mouse i at time t, β₀ is the intercept representing the log ratio of the day 26 tumor volume to the initial volume for the control group, β₁ is the linear effect of time for the control group, β₂ is the group effect on day 26, β₃ is the group effect in terms of the linear effect of time. The mouse specific effect, ζ₀₁, represented the deviation of each mouse from the group intercept. This corrected for the correlation between measurements taken on the same mouse. Since the mice in this experiment were considered a representative sample from a larger population, the effect was considered random and it was assumed that the population distribution from which they were sampled had a normal distribution.

Student's t-tests were performed to assess for statistically significant differences in the percentage of cells that stained positively for phosphorylated histone H3 and phosphorylated aurora A-C.

The tumors were measured with a digital caliper and the mice were weighed twice per week. Tumor volumes were calculated as follows: Tumor Volume (mm³)=[(W²×L)/2] where width (W) is defined as the smaller of the 2 measurements and length (L) is defined as the larger of the 2 measurements. Tumor inhibition was calculated as follows: first; take [Initial tumor volume minus final tumor volume] for control and all treatment groups; second, take the change in treated tumor volume divided by control tumor volume minus one and then multiply by 100. FIG. 6 hereinbelow describe the tumor volume and tumor growth results.

Combination treatment with AMG 900 and SAHA surprisingly resulted is synergistically significant tumor growth inhibition using both: doses of AMG 900 compared with the single agent group and vehicle control group (FIG. 6).

### Example 4

In addition, in-vivo expression of the molecular marker pH3 was measured, after AMG 900 and/or SAHA treatment, alone or in combination.

### In Vivo Method

The Animal protocol preparation of DU-145 prostate cells, agent formulation, measuring and analysis of tumor growth, tissue immunostaining and statistical methods are identical to that described in Example 3 hereinabove.

FIG. 7 illustrates the results, which show that levels of pH3 are decreased in mice with DU-145 tumors after treatment with a low dose of AMG 900 in combination with treatment with SAHA. FIG. 7 further illustrates that the low dose effect is at least comparable, and maybe even superior, to single agent AMG 900 treatment at the higher dose.

### INDICATIONS

AMG 900 is a pan aurora kinase inhibitor. Aurora kinase proteins play a part in cell cycling and, therefore, cell proliferation. Aurora kinases are enzymes of the serine/threonine kinase family of proteins, which play an important role in protein phosphorylation during the mitotic phase of the cell cycle. There are three known members of the Aurora kinase family, Aurora A, Aurora B and Aurora C, also referred to as Aurora 2, Aurora 1, and Aurora 3, respectively.

Specifically, the function of each Aurora kinase isoform in mammalian cell cycle has been studied. Aurora-A is localized to the centrosome during interphase and is important for centrosome maturation and to maintain separation during spindle assembly. Aurora-B localizes to the kinetochore in the G2 phase of the cell cycle until metaphase, and relocates to the midbody after anaphase. Aurora-C was thought to function only in meiosis, but more recently has been found to be more closely related to Aurora-B, showing some overlapping functions and similar localization patterns in mitosis. Each aurora kinase appears to share a common structure, including a highly conserved catalytic domain and a very short N-terminal domain that varies in size. (See R. Giet and C. Prigent, J. Cell Sci., 1123591-3601 (1999)).

Aurora kinases are over expressed in various types of cancers, including colon, breast, lung, pancreas, prostate, bladder, head, neck, cervix, and ovarian cancers. The Aurora-A gene is part of an amplicon found in a subset of breast, colon, ovarian, liver, gastric and pancreatic tumors. Aurora-B has also been found to be over expressed in most major tumor types. Over expression of Aurora-B in rodent fibroblasts induces transformation, suggesting that Aurora-B is oncogenic. More recently, Aurora-B mRNA expression has been linked to chromosomal instability in human breast cancer. (Y. Miyoshi et al., Int. J. Cancer, 92:370-373 (2001)).

As stated hereinbefore, HDAC inhibitors can induce p21 (WAF1) expression, a regulator of p53's tumor suppressor activity. HDACs are involved in the pathway by which the retinoblastoma protein (pRb) suppresses cell proliferation. The pRb protein is part of a complex that attracts HDACs to the chromatin so that it will deacetylate histones. HDACl negatively regulates the cardiovascular transcription factor Kruppel-like factor 5 through direct interaction. Estrogen is well-established as a mitogenic factor implicated in the tumorigenesis and progression of breast cancer via its binding to the estrogen receptor alpha (ERα). Recent data indicate that chromatin inactivation mediated by HDAC and DNA methylation is a critical component of ERα silencing in human breast cancer cells.

The present invention provides a method of treating cancer by administering an aurora kinase inhibitor compound, AMG 900, in combination with HDAC inhibiting agent, such as presently approved HDACs for medical use by regulatory agencies, including without limitation, Vorinostat and Romidepsin, and HDAC agents in clinical, trials, including without limitation Panobinostat (LBH589), Valproic acid (as Mg valproate); Belinostat (PXD101), Mocetinostat (MGCD103), Abexinostat (PCI-24781), Entinostat (MS-275), SB939, Resminostat (4SC-210), Givinostat CUDC-101, AR-42, CHR-2845, CHR-3996, 4SC,202 , CG200745, ACY-1215 and Sulforaphane. The invention further provides AMG 900 in combination with other pre-clinical HDAC inhibitors in the treatment of cancer.

In vitro treatment of a panel of prostate cancer cell lines with low-dose combinations of AMG 900 with HDACIs revealed enhanced antiproliferative responses compared to single agent use. Short term MTS assays only demonstrated moderate synergism in DU-145 and LNCaP cells co-treated with VPA and synergism in PC3 cells co-treated with vorinostat, based on the Chou-Talalay-method (CalcuSyn). However, all cell lines demonstrated significant decreased long term clonogenic survival when combining low-dose AMG 900 (1 nM) with HDACIs. In DU-145 and PC3 cells lines, the low-dose AMG 900 (1 nM) combinations demonstrated equivalent clonogenic survival to high dose AMG 900 treatment (5 nM), either alone or in combination with HDACIs. Morphologic changes were noted in treated cell lines sod senescence was explored with beta-galactosidase staining. DU-145 and PC3 cells, treated with a combination of AMG 900 and HDACIs, demonstrated a significant increase in beta-galactosidase positive cells, suggesting cell senescence (10-20% in single agent vs 50% in low-dose combinations).

The mechanisms associated with decreased clonogenic survival were also explored at low dose combinations and a significant increase in cleaved PARP in DU-145 cells with both combinations, and with vorinostat in LNCaP cells, was observed but not in PC3 cells which have a baseline level of ongoing apoptosis. In addition, Western blot analysis showed significant differences in protein expression levels of phosphorylated aurora, phosphorylated Histone H3, and p21 in combination treatment compared to single agent use. In vivo xenograft DU-145 model demonstrated low dose combination of AMG 900 and SAHA was equivalent to high dose AMG 900 alone. To this end, a combination of low-dose AMG 900 with HDACIs in prostate cancer cell lines demonstrated unexpected synergy and efficacy equivalent to high doses of AMG 900 alone.

Accordingly, in embodiment 1 of the invention, there is provided a method of treating cancer in a subject, the method comprising administering to the subject an effective dosage amount of AMG 900, or a pharmaceutically acceptable salt thereof, in combination with an HDAC inhibiting agent wherein the combined therapy treats the cancer.

In embodiment 2 of the invention, there is provided the method of embodiment 1 wherein the HDAC inhibiting agent is Vorinostat, Romidepsin, Panobinostat (LBH589), valproic acid, Belinostat (PXD101), Mocetinostat (MGCD103), Abexinostat(PCI-24781), Entinostat (MS-275), SB939, Resminostat (4SC-210), Givinonstat (ITF2357), CUDC-101, AR-42, CHR-2845, CHR-3996, 4SC-202, CG200745, ACY-1215 or Sulforphane.

In embodiment 3 of the invention, there is provided the method of any one of embodiments 1 and 2 wherein the HDAC inhibiting agent is Vorinostat, Romidepsin, Panobinostat (LBH589), valproic acid, Belinostat (PXD101), Mocetinostat (MGCD103), Abexinostat (PCI-24781), Entinostat (MS-275), SB939, Resminostat (4SC-210), Givinostat (ITF2357).

In embodiment 4 of the invention, there is provided the method of any one of embodiments 1-3 wherein the HDAC inhibiting agent is Vorinostat, Romidepsin, Panobinostat (LBH589) or valproic acid.

In embodiment 5 of the invention, there is provided the method of any one of embodiments 1-4 wherein the HDAC inhibiting agent is Vorinostat, Romidepsin or valproic acid.

In embodiment 6 of the invention, there is provided the method of any one of embodiments 1-5 wherein the HDAC inhibiting agent is Vorinostat.

In embodiment 7 of the invention, there is provided the method of any one of embodiments 1-6 wherein the cancer is one or more of (a) a solid or hematologically derived tumor selected from (a) cancer of the bladder, breast, colon, kidney, liver, lung, small cell long cancer, esophagus, gall-bladder, ovary, pancreas, stomach, cervix, thyroid, prostate and skin, (b) a hematopoietic tumor of lymphoid lineage selected from leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma, (c) a hematopoietic tumor of myeloid lineage selected from acute and chronic myelogenous leukemias, myelodysplastic syndrome and promyelocytic leukemia (d) a tumor of mesenchymal origin selected from fibrosarcoma and rhabdomyosarcoma, (e) a tumor of the central and peripheral nervous system selected from astrocytoma, neuroblastoma, glioma and schwannoma, and (f) a melanoma, seminoma, teratocarcinoma, osteosarcoma, xenoderoma pigmentosum, keratoctanthoma, thyroid follicular cancer or Kaposi's sarcoma.

In embodiment 8 of the invention, there is provided the method of any one of embodiments 1-6 wherein the cancer is one or more of a solid tumor selected from cancer of the bladder breast, colon, kidney, liver, lung, non-small cell lung, head and neck, esophageal, gastric, ovary, pancreas, stomach, cervix, thyroid and prostate or a lymphoma or leukemia.

In embodiment 9 of the invention, there is provided the method of any one of embodiments 1-6 wherein the cancer is prostate cancer, ovarian cancer, breast cancer, cholangiocarcinoma, acute myeloid leukemia, chronic myeloid leukemia or a combination thereof.

In embodiment 10 of the invention, there is provided the method of any one of embodiments 1-9 wherein the effective dosage amount of AMG 900 or a pharmaceutically acceptable salt thereof, is in the range of about 0.5 mg/kg to about 30 mg/kg weight of the subject.

In embodiment 11 of the invention, there is provided the method of any one of embodiments 1-9 wherein effective dosage amount of AMG 900 or a pharmaceutically acceptable salt thereof, is in the range of about 2.5 mg/kg to about 24 mg/kg weight of the subject.

In embodiment 12 of the invention, there is provided the method of any one of embodiments 1-9 wherein the effective dosage amount of AMG 900 or a pharmaceutically acceptable salt thereof is in the range of about 2.5 mg/kg to about 10 mg/kg weight of the subject.

In embodiment 13 of the invention, there is provided a method of reducing the size of a solid tumor in a subject, the method comprising administering to the subject an effective dosage amount of the compound AMG 900, or a pharmaceutically acceptable salt thereof, in combination with an HDAC inhibiting agent, wherein the combined therapy reduces the size of the tumor.

In embodiment 14 of the invention, there is provided the method of any one of embodiments 1-12 wherein the AMG 900, or a pharmaceutically acceptable salt thereof, and the HDAC inhibiting agent are administered the same day.

In embodiment 15 of the invention, there is provided a method of any one of embodiments 1-12 wherein the AMG 900, or a pharmaceutically acceptable salt thereof, and the HDAC inhibiting agent are administered sequentially or co-administered simultaneously.

In embodiment 16 of the invention, there is provided a method of any one of embodiments 1-12 wherein the AMG 900, or a pharmaceutically acceptable salt thereof, and the HDAC inhibiting agent are co-administered in a single dosage formulation.

In embodiment 17 of the invention, there is provided a method of any one of embodiments 1-12 wherein the AMG 900, or a pharmaceutically acceptable salt thereof, and the HDAC inhibiting agent are co-administered as separate dosage formulations.

In embodiment 18 of the invention, there is provided a method of any one of embodiments 13-17 wherein the HDAC inhibiting agent is Vorinostat, Romidepsin, Panobinostat (LBH589), valproic acid, Belinostat (PXD101), Mocetinostat (MGCD103), Abexinostat (PCI-24781), Entinostat (MS-275), SB939, Resminostat (4SC-210), Givinostat (ITF2357), CUDC-101, AR-42, CHR-2845, CHR-3996, 4SC-202, CG200745, ACY1215 or Sulforphane.

To this end, the combination treatment of AMG 900 with an HDAC inhibiting agent is useful for, but not limited to, the prevention or treatment of cancer including, for example, various solid and hematologically derived tumors, such as carcinomas, including, without limitation, cancer of the bladder, breast, colon, kidney, liver, lung (including small cell lung, cancer), esophagus, gall-bladder, ovary, pancreas, stomach, cervix, thyroid, prostate, uterus and skin (including squamous cell carcinoma); hematopoietic tumors of lymphoid lineage (including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma); hematopoietic tumors of myeloid lineage (including acute and chronic myelogenous leukemias (AML and CML), myelodysplastic syndrome and promyelocytic leukemia); tumors of mesenchymal origin (including fibrosarcoma and rhabdomyosarcoma, and other sarcomas, e.g. soft tissue and bone); tumors of the central and peripheral nervous system (including astrocytoma, neuroblastoma, glioma and schwannomas; and other tumors (including melanoma, seminoma, teratocarcinoma, osteosarcoma, xenoderoma pigmentosum, keratoctanthoma, thyroid follicular cancer and Kaposi's sarcoma), where such cancers have relapsed or become refractory. Cancers, such as prostate cancer, ovarian cancer, lung cancer, breast cancer, cholangiocarcinoma or other types of cancer, which have become refractory to anti-cancer treatment, such as with hormones, may also be treated with AMG 900.

For example, AMG 900 can be used in combination with an HDAC inhibitor, such as vorinostat or valprioc acid, to treat breast cancer (Siegel D., J. Hematologic Oncology, 2:31, 2009). Triple negative breast cancer is an aggressive form of breast cancer characterized by the lack of hormone receptors, estrogen and progesterone receptors, and HER2/neu expression, for which there are no effective treatment options.

In embodiment 19, the invention provides the method of any one of embodiments 1-17 wherein the cancer treated is a solid tumor selected from cancer of the bladder, breast, colon, kidney, liver, lung, non-small cell lung, head and neck, esophageal, gastric, ovary, pancreas, stomach, cervix, thyroid and prostate cancer.

In embodiment 20, the invention provides the method of any one of embodiments 1-17 wherein the cancer treated is prostate cancer.

In embodiment 21, the invention provides the method of any one of embodiments 1-17 wherein the HDAC inhibiting agent is Vorinostat, Romidepsin, Panobinostat (LBH-589), valproic acid, Belinostat (PXD101), Mocetinostat (MGCD103), Abexinostat (PCI-24781), Entinostat (MS-275), SB939, Resminostat (4SC-210), Givinostat (ITF2357), CUDC-101, AR-42, CHR-2845, CHR-3996, 4SC-202, CG200745, ACY-1215 or Sulforphane and the cancer treated is a solid tumor selected from cancer of the bladder, breast, colon, kidney, liver, lung, non-small cell lung, head and neck, esophageal, gastric, ovary, pancreas, stomach, cervix, thyroid prostate cancer.

In embodiment 22, the invention provides the method of any one of embodiments 1-19 and 21 wherein the HDAC inhibiting agent is Vorinostat of valproic acid and the cancer treated is a solid tumor selected from cancer of the bladder, breast, colon, kidney, liver, lung, non-small cell lung, head and neck, esophageal, gastric, ovary, pancreas, stomach, cervix, thyroid and prostate cancer.

In embodiment 23, the invention provides the method of any one of embodiments 1-20 wherein the HDAC inhibiting agent is Vorinostat or valproic acid and the cancer treated is prostate cancer.

In embodiment 24, the invention provides the method of any one of embodiments 1-19, 21-22 wherein the HDAC inhibiting agent is Vorinostat or valproic acid and the cancer treated is breast cancer.

In embodiment 25, the invention provides the method of any one of embodiments 1-19, 21-22 and 24 wherein the HDAC inhibiting agent is Vorinostat or valproic acid and the cancer treated is triple negative breast cancer.

The invention also provides a method for the treatment of solid tumors, sarcomas (especially Ewing's sarcoma and osteosarcoma), retinoblastoma, rhabdomyosarcomas, neuroblastoma, hematopoietic malignancies, including leukemia and lymphoma, tumor-induced pleural or pericardial effusions, and malignant ascites.

Besides being useful for human treatment, the compound is also useful for veterinary treatment of companion animals, exotic animals and farm animals, including mammals, rodents, and the like. For example, animals including horses, dogs, and cats may be similarly treated with a combination of AMG 900 and HDAV inhibitors for cancers.

### FORMULATIONS

Each of AMG 980 and the HDAC inhibiting agent may be administered to the cancer subject in combination as a single or separate pharmaceutical compositions or medicaments, comprising the active pharmaceutical ingredient (API), ie., AMG 900 (N-(4-((3-(2-amino-4-pyrimidinyl-2-pyridinyl)oxy)phenyl)-4-(4-methyl-2-thienyl)-1-phthalazinamine) and the HDAC inhibiting agent, in association with one or more non-toxic, pharmaceutically-acceptable carriers, diluents and/or adjuvants (collectively referred to herein as “excipient” materials). Both AMG 900, or a pharmaceutically acceptable salt form thereof, and the HDAC inhibitor API can be processed in accordance with conventional methods of pharmacy to produce the medicinal and pharmaceutical compositions for administration to patients, including humans and other mammals.

The pharmaceutical composition may be administered to the subject by any suitable route, adapted to such a route, and in a dose effective for the refractory cancer treatment intended. The composition, or API, may for example, be administered orally, mucosally, topically, rectally, pulmonarily such as by inhalation spray, or parentally including intravascularly, intravenously, intraperiotoneally, subcutaneously, intramuscularly intrasternally and infusion techniques, in dosage unit formulations containing conventional pharmaceutically acceptable carriers, adjuvants, and vehicles.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. For example, these may contain an amount of active ingredient from about 1 to 2000 mg, and typically from about 1 so 500 mg. A suitable daily dose for a human or other mammal may vary widely depending on the condition of the patient and other factors, but, once again, can be determined using routine methods and practices.

The amount of the API (AMG 900) which is administered and the dosage regimen for treating the refractory cancer condition depends on a variety of factors, including the age, weight, sex and medical condition of the subject, the type of disease, the severity of the cancer, the route and frequency of administration, and the physical and chemical properties of AMG 900 or its particular form, including the specific salt form. Thus, a dosage regimen may vary. A daily dose of about 0.01 to 500 mg/kg, advantageously between about 0.01 and about 50 mg/kg, more advantageously about 0.1 and about 30 mg/kg and even more advantageously between about 0.1 mg/kg and about 25 mg/kg body weight may be appropriate. In one embodiment, the invention provides a method of treating cancer in a subject, the method comprising administering to the subject AMG 900 or a pharmaceutically acceptable salt thereof in an effective dosage amount in the range from about 0.5 mg/kg to about 25 mg/kg, wherein the subject's cancer is refractory to treatment with an anti-mitotic agent. In another embodiment, the invention provides a method of treating cancer in a subject, the method comprising administering, to the subject AMG 900 or a pharmaceutically acceptable salt thereof in an effective dosage amount in the range from about 1.0 mg/kg to about 20 mg/kg, wherein the subject's cancer is refractory to treatment with standard of care chemotherapeutic agent, including an anti-mitotic agent. In yet another embodiment, the invention provides a method of treating cancer in a subject, the method comprising administering to the subject AMG 900 or a pharmaceutically acceptable salt thereof in an effective dosage amount in the range from about 3.0 mg/kg to about 15 mg/kg, wherein the subject's cancer is refractory to treatment with an anti-mitotic agent. The daily dose can be administered in one to four doses per day.

For therapeutic purposes, AMG 900 may be combined with one or more adjuvants or “excipients” appropriate to the indicated route of administration. If administered on a per dose basis, AMG 900 may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, to form the final formulation. For example, AMG 900 and the excipient(s) may be tableted or encapsulated by known and accepted methods for convenient administration. Examples of suitable formulations include, without limitation, pills, tablets, soil and hard-shell gel capsules, troches, orally-dissolvable forms and delayed or controlled-release formulations thereof. Particularly, capsule or tablet formulations may contain one or more controlled-release agents, such as hydroxypropylmethyl cellulose, as a dispersion with the API(s).

In the case of psoriasis and other skin conditions, it may be preferable to apply a topical preparation of the AMG 900 to the affected area two to four times a day. Formulations suitable for topical administration include liquid or semiliquid preparations suitable for penetration through the skin (e.g., liniments, lotions, ointments, creams, pastes, suspensions and the like) and drops suitable for administration to the eye, ear, or nose. A suitable topical dose of the active ingredient as 0.3 mg to 150 mg administered one to four, preferably one or two times daily. For topical administration, the API may comprise from 0.001% to 10% w/w, e.g., from 1% to 2% by weight of the formulation, although it may comprise as much as 10% w/w, but preferably not more than 5% w/w, and more preferably from 0.1 to 1% of the formulation.

When formulated in an ointment, AMG 900 may be employed with either paraffinic or a water-miscible ointment base. Alternatively, it may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound, which enhances absorption or penetration of the active ingredient through the skin or other affected areas. Examples of such dermal penetration enhancers include DMSO and related analogs.

AMG 900 can also be administered by transdermal device. Preferably transdermal administration will be accomplished using a patch either of the reservoir and porous membrane type or of a solid matrix variety. In either case, AMG 900 is delivered continuously from the reservoir or microcapsules through a membrane into the active agent permeable adhesive, which is in contact with the skin or mucosa of the recipient. If AMG 900 is absorbed through the skin, a controlled and predetermined flow of AMG 900 is administered to the recipient. In the case of microcapsules the encapsulating agent may also function as the membrane.

The oily phase of the emulsions may be constituted from known ingredients in a known manner. While the phase may comprise merely an emulsifier, it may comprise a mixture of at least one emulsifier with a fat, or an oil, or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base, which forms the oily dispersed phase of the cream formulations. Emulsifiers and emulsion stabilizers suitable for use in the formulation include, for example, Tween 60, Span 80, cetostearyl alcohol, myristyl alcohol, glyceryl monostearase, sodium lauryl sulfate, glyceryl distearate alone or with a wax, or other materials well known in the art.

The choice of suitable oils or fats for the formulation is based on achieving the desired cosmetic properties, since the solubility of the API in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable manner, especially an aqueous solvent for AMG 900. AMG 900 is preferably present in such formulations in a concentration of 0.5 to 20%, advantageously 0.5 to 10% and particularly about 1.5% w/w.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. For example AMG 900 may be dissolved in water, polyethylene glycol, propylene glycol, ethanol, corn oil cottonseed oil, peanut oil, sesame oil, benzyl alcohol, sodium chloride, tragacanth gum, and/or various buffers. Other adjuvants and modes of administration are well and widely known in the pharmaceutical art. AMG 900 may also be administered by injection as a composition with suitable carriers including saline, dextrose, or water, or with cyclodextrin (ie. Captisol), cosolvent solubilization (ie. propylene glycol) or micellar solubilization (ie. Tween 80).

The sterile injectible preparation may also be a sterile injectible solution or suspension in a non-toxic parenterally acceptable diluent or solvent, for example as a solution in 1,3-butanediol. Among the acceptable vehicles and solvents that may be employed are water, Ringers solution, and isotonic sodium chloride solution. In addition, sterile, fixed oils are conventionally employed as a solvent or suspending medium. For this purpose any bland fixed oil may be employed, including synthetic mono- or diglycerides. Further, fatty acids such as oleic acid find use in the preparation of injectables.

For pulmonary administration, the pharmaceutical composition may be administered in the form of an aerosol or with an inhaler including dry powder aerosol.

Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug.

The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

### COMBINATION ADMINISTRATION

The present invention provides AMG 900 in combination with an HDAC inhibitor for the treatment of cancer. Thus, each of the AMG 900 and the HDAC inhibitor independently can be formulated as separate compositions, such as a tablet, capsule or injectable solution, which is individually administered simultaneously or sequentially at different times. Or both API's (AMC 900 and the HDAC Inhibitor) can be formulated as a single formulation, such as a unit tablet, capsule or injectable solution comprising both API's, and co-administered in a substantially simultaneous manner as a single composition. The invention includes a single capsule having a fixed ratio of both active agents or multiple, separate capsules for each agent.

The phrase “co-administration”, “co-therapy” or “combination-therapy”, in defining the use of AMG 900 in the present invention is intended to embrace both modes of administration of the drug combination, ie., either as a single formulation simultaneously administered or as separate formulations administered in a sequential manner in a regimen that will provide beneficial effects.

The foregoing is merely illustrative of the invention and is not intended to limit the invention to the disclosed uses. Variations and changes, which are routine to one skilled in the art, are intended to be within the scope and nature of the invention, which are defined in the amended claims. All mentioned references, patents, applications and publications, are hereby incorporated by reference in their entirety, as if here written.

1. A method of treating cancer in a subject comprising administering to the subject an effective dosage amount of AMG 900, or a pharmaceutically acceptable salt thereof, in combination with an HDAC inhibiting agent, wherein the combined therapy treats the cancer.

2. The method of claim 1, wherein the HDAC inhibiting agent is selected from the group consisting of Vorinostat, Romidepsin, Panobinostat (LBH589), valproic acid, Belinostat (PXD101), Mocetinostat (MGCD103), Abexinostat (PCI-24781), Entinostat (MS-275), SB939, Resminostat (4SC-210), Givinostat (ITF2357), CUDC-101, AR-42, CHR-2845, CHR-3996, 4SC-202, CG200745, ACY-1215 and Sulforphane.

3.-5. (canceled)

6. The method of claim 2, wherein the HDAC inhibiting agent is Vorinostat.

7. The method of claim 2, wherein the cancer is one or more of (a) a solid or hematologically derived tumor selected from the group consisting of (a) cancer of the bladder, breast, colon, kidney, liver, lung, small cell lung cancer, esophagus, gall-bladder, ovary, pancreas, stomach, cervix, thyroid, prostate and skin, (b) a hematopoietic tumor of lymphoid lineage selected from leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma, (c) a hematopoietic tumor of myeloid lineage selected from acute and chronic myelogenous leukemias, myelodysplastic syndrome and promyelocytic leukemia (d) a tumor of mesenchymal origin selected from fibrosarcoma and rhabdomyosarcoma, (e) a tumor of the central and peripheral nervous system selected from astrocytoma, neuroblastoma, glioma and schwannoma, and (f) a melanoma, seminoma, teratocarcinoma, osteosarcoma, xenoderoma pigmentosum, keratoctanthoma, thyroid follicular cancer and Kaposi's sarcoma.

8.-9. (canceled)

10. The method of claim 2, wherein the effective dosage amount of AMG 900 or a pharmaceutically acceptable salt thereof, is in the range of about 0.5 mg/kg to about 30 mg/kg weight of the subject.

11. The method of claim 2, wherein the effective dosage amount of AMG 900 or a pharmaceutically acceptable salt thereof, is in the range of about 2.5 mg/kg to about 24 mg/kg weight of the subject.

12. The method of claim 2, wherein the effective dosage amount of AMG 900 or a pharmaceutically acceptable salt thereof, is in the range of about 2.5 mg/kg to about 10 mg/kg weight of the subject.

13. A method of reducing the size of a solid tumor in a subject, the method comprising administering to the subject an effective dosage amount of the compound AMG 900, or a pharmaceutically acceptable salt thereof, in combination with an HDAC inhibiting agent, wherein the combined therapy reduces the size of the tumor.

14. The method of claim 13, wherein the AMG 900, or a pharmaceutically acceptable salt thereof, and the HDAC inhibiting agent are administered the same day.

15. The method of claim 13, wherein the AMG 900, or a pharmaceutically acceptable salt thereof, and the HDAC inhibiting agent are administered sequentially or co-administered simultaneously.

16. The method of claim 13, wherein the AMG 900, or a pharmaceutically acceptable salt thereof, and the HDAC inhibiting agent are co-administered in a single dosage formulation.

17. The method of claim 13, wherein the AMG 900, or a pharmaceutically acceptable salt thereof, and the HDAC inhibiting agent are co-administered as separate dosage formulations.

18. The method of claim 13, wherein the HDAC inhibiting agent is selected from the group consisting of: Vorinostat, Romidepsin, Panobinostat (LBH589), valproic acid, Belinostat (PXD101), Mocetinostat (MGCD103), Abexinostat (PCI-24781), Entinostat (MS-275), SB939, Resminostat (4SC-210), Givinostat (ITF2357), CUDC-101, AR-42, CHR-2845, CHR-3996, 4SC-202, CG200745, ACY-1215 and Sulforphane.
